(19) United States
(12) Patent Application Publication
(10) Pub. No.: US 2025/0064606 A1
(43) Pub. Date: Feb. 27, 2025

(54) METHODS, SYSTEMS, APPARATUSES, AND DEVICES FOR FACILITATING CONTROLLING OF A DEVICE

(71) Applicant: Manar Al Harakeh, Achrafieh (LB)

(72) Inventor: Manar Al Harakeh, Achrafieh (LB)

(21) Appl. No.: 18/812,816

(22) Filed: Aug. 22, 2024

Related U.S. Application Data
(60) Provisional application No. 63/578,120, filed on Aug. 22, 2023.

(51) Int. Cl. A61F 2/72; A61F 2/54; A61F 2/58; A61F 2/68; A61F 2/70; A61F 2/80; A63F 13/211; A63F 13/212; G06F 3/01

(52) U.S. Cl. CPC A61F 2/72; A61F 2/583; A61F 2/585; A61F 2/586; A61F 2/80; A63F 13/212; A61F 2002/543; A61F 2002/6881; A61F 2002/689; A61F 2002/704; A63F 13/211; G06F 3/015

(57) ABSTRACT

Disclosed herein is a system for facilitating controlling of a device. Accordingly, the system may include an Electromyographic (EMG) sensor configured to be attached to a residual limb and a part of a body of. Further, the EMG sensor may be configured to generate an EMG data based on an activity of a muscle of the user. Further, the system may include a processing device communicatively coupled with the EMG sensor. Further, the processing device may be configured to analyze the EMG data, determine an EMG pattern associated with the EMG data based on the analysis, and generate an action data based on the EMG pattern. Further, the system may include a communication device communicatively coupled with the processing device. Further, the communication device may be configured to transmit the action data to at least one of a controllable device and a prosthetic device.

### FIELD OF THE INVENTION

Generally, the present disclosure relates to the field of portable electronic devices (for example, tablets and smartphones) and body-borne computers or wearable devices. More specifically, the present disclosure relates to methods, systems, apparatuses, and devices for facilitating controlling of a device.

### BACKGROUND OF THE INVENTION

The field of portable electronic devices (for example, tablets and smartphones) and body-borne computers or wearable devices is technologically important to several industries, business organizations, and/or individuals.

The advent of prosthetic technology has significantly transformed the lives of individuals with limb loss or limb impairments. Traditional prosthetic hands have primarily focused on restoring basic functionality, such as grasping and holding objects. However, as technology continues to advance, there is an increasing demand for prosthetics that offer not only functional improvement but also enhanced interaction with modern digital environments. Smartphones, tablets, computers, and a myriad of smart devices now utilize Bluetooth connectivity for seamless communication and control. As such, there is a growing need for innovative solutions that enable users to interact with these devices in a more intuitive and efficient manner. Current prosthetic devices, while effective in providing basic hand functions, often lack the capability to integrate with modern wireless technologies. This gap creates a limitation for the users who wish to control Bluetooth-enabled devices using their prosthetics. Existing techniques for facilitating controlling of a device are deficient with regard to several aspects. For instance, current technologies do not use electromyography sensors for detecting the gestures to control the Bluetooth device. Furthermore, current technologies do not use machine learning algorithms to identify gestures and generate a command for Bluetooth devices. Moreover, current technologies do not provide a mode button to allow a user to use the bionic hand as a control device such as a game controller or an AR/VR controller.

Therefore, there is a need for improved methods, systems, apparatuses, and devices for facilitating controlling of a device that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a system for facilitating controlling of a device, in accordance with some embodiments. Accordingly, the system may include an Electromyographic (EMG) sensor configured to be attached to one or more of a residual limb of a user and a part of a body of the user. Further, the part of the body may be proximal to a limb of the user. Further, the EMG sensor may be configured to generate an EMG data based on an activity of a muscle of the user. Further, one or more of the residual limb and the part may include the muscle. Further, the system may include a processing device communicatively coupled with the EMG sensor. Further, the processing device may be configured to analyze the EMG data. Further, the processing device may be configured to determine an EMG pattern associated with the EMG data based on the analysis. Further, the EMG pattern corresponds to an intended action of the user. Further, the processing device may be configured to generate an action data based on the EMG pattern. Further, the action data may include an indication of the intended action of the user. Further, the system may include a communication device communicatively coupled with the processing device. Further, the communication device may be configured to transmit the action data to at least one of a controllable device and a prosthetic device.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a block diagram of a system 100 for facilitating controlling of a device, in accordance with some embodiments.

FIG. 2 illustrates the prosthetic device 110 for facilitating controlling of the device, in accordance with some embodiments.

FIG. 3 is an exploded view of the prosthetic device 110 for facilitating controlling of the device, in accordance with some embodiments.

FIG. 4 is a block diagram of the system 100 for facilitating controlling of the device, in accordance with some embodiments.

FIG. 5 is a block diagram of the system 100 for facilitating controlling of the device, in accordance with some embodiments.

FIG. 6 is a block diagram of the system 100 for facilitating controlling of the device, in accordance with some embodiments.

FIG. 7 is a block diagram of the system 100 for facilitating controlling of the device, in accordance with some embodiments.

FIG. 8 is an illustration of an online platform consistent with various embodiments of the present disclosure.

FIG. 9 is a block diagram of a system 900 for facilitating controlling devices based on gesture recognition using a bionic hand 908, in accordance with some embodiments.

FIG. 10 is a flow chart of a method 1000 for facilitating controlling devices based on gesture recognition using a bionic hand, in accordance with some embodiments.

FIG. 11 illustrates an EMG electrode board 1100 comprised in a system for facilitating controlling the devices based on gesture recognition, in accordance with some embodiments.

FIG. 12 illustrates a sensor board 1200 of the system for facilitating controlling the devices based on gesture recognition, in accordance with some embodiments.

FIG. 13 is a block diagram of a main board 1300 for facilitating controlling the devices based on gesture recognition, in accordance with some embodiments.

FIG. 14 is a flow diagram of a method 1400 for facilitating controlling the devices based on gesture recognition using the bionic hand, in accordance with some embodiments.

FIG. 15 is a continuation flow diagram of FIG. 14.

FIG. 16 is a continuation flow diagram of FIG. 14.

FIG. 17 is a continuation flow diagram of FIG. 14.

FIG. 18 is a block diagram of a computing device for implementing the methods disclosed herein, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, systems, apparatuses, and devices for facilitating controlling of a device, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosures describes methods, systems, apparatuses, and devices for facilitating controlling of a device. Further, Geko hand, an exemplary embodiment of a prosthetic device (or a bionic hand) herein, may include a prosthetic hand that allows users to control Bluetooth-enabled devices using gestures detected by eight electromyography (EMG) sensors.

Further, the bionic hand enables individuals with limb loss or limb impairments to regain dexterity and control over Bluetooth devices through intuitive hand gestures. Further, the bionic hand may combine advanced gesture recognition technology with the capability to wirelessly interact with various Bluetooth-enabled devices like a computer mouse or a gamepad. Further, the bionic hand may act as a mouse, a gamepad controller, or a VR controller. Furthermore, on top of using gestures to control one or more motors of the bionic hand, the user may map the gestures to control a mouse pointer and perform mouse computer tasks. Further, the same goes for the gamepad and VR controller. Additionally, the gestures may be mapped to specific buttons allowing the user to interact with the Bluetooth device.

Further, the bionic hand works by capturing and interpreting the electrical signals generated by the user's muscles through EMG sensors. The sensors may be strategically placed on the user's residual limb or other muscles in proximity. When the user thinks about performing a specific hand movement, the corresponding muscles generate unique electrical patterns. The EMG sensors detect these patterns, convert them into digital signals, and transmit the information to the bionic hand's control system.

Further, the bionic hand may include a control system that processes the incoming signals from the EMG sensors using advanced algorithms and machine learning techniques. The algorithms analyze the patterns in real-time and determine the intended movement based on pre-trained models. Once the intended movement is identified, the control system commands the bionic hand's motors to execute the desired motion.

Additionally, the bionic hand is paired with a software application installed on a smartphone or PC via Bluetooth. The application provides an interface that allows the users to control Bluetooth-enabled devices, such as a mouse or a gamepad, using the bionic hand's movements and/or gestures performed by the residual limp that may be analyzed by the gesture recognition algorithm. Moreover, the application may allow the user to control the bionic hand. Furthermore, the application may allow the user to train machine learning algorithms. The application receives the signals from the bionic hand's control system, translates them into appropriate commands, and wirelessly transmits them to the target Bluetooth devices, enabling seamless control.

Further, the disclosed bionic hand may include a prosthetic hand comprising a physical artificial hand with motorized fingers and a wrist joint.

Further, the bionic hand may include eight EMG sensors strategically placed on the user's limb or muscles. Further, the bionic hand may include a signal processing unit comprising hardware and software components responsible for processing and analyzing the EMG signals. Further, the bionic hand may be based on a gesture recognition algorithm that interprets the EMG signals to identify specific hand gestures. Further, the bionic hand may include a wireless communication module such as a Bluetooth transmitter that transmits the gesture commands to the paired application.

Further, the bionic hand may be associated with a user interface that allows users to pair and manage the Bluetooth devices and customize gesture mappings. Further, the bionic hand may be configured for performing Gesture-to-Action Mapping using software logic that maps specific gestures to predefined actions for the different Bluetooth devices. Further, the bionic hand may include a Bluetooth controller module responsible for establishing and managing Bluetooth connections with target devices. Further, the bionic hand may be based on software algorithms that control the target Bluetooth devices based on the received gesture commands. The EMG sensors are connected to the signal processing unit of the bionic hand, which processes the electrical signals captured by the sensors. The processed signals may be then fed into a gesture recognition algorithm, that identifies the user's hand gestures based on predefined patterns or machine learning techniques. Once a gesture is recognized, gesture commands may be wirelessly transmitted to the paired application using the Bluetooth transmitter in the bionic hand. The application receives the commands and interprets them based on the customized gesture-to-action mapping. Further, the application may then communicate with the target Bluetooth devices, such as a computer or gamepad, using the Bluetooth controller module. The application's device control logic translates the gesture commands into specific actions, enabling the user to perform tasks like moving the mouse cursor, clicking buttons, or controlling gamepad inputs. The feedback from the application, such as visual or haptic feedback, may be relayed back to the user through the bionic hand if desired. Overall, the components of the bionic hand and the paired application work together to detect hand gestures using EMG sensors, interpret them into meaningful commands, and control Bluetooth devices wirelessly, providing the user with enhanced functionality and control over their digital environment.

Further, in some embodiments, the bionic hand connects to a Bluetooth enabled device (a personal computer for example) via Bluetooth as a computer mouse allowing the user to interact with the personal computer using their performed gestures. The same goes for other devices. Further, the bionic hand may connect with a PC as a gamepad, allowing the user to play games and interact with games using gestures as buttons.

The bionic hand may act as any input Bluetooth device (mouse, gamepad, VR controller, presentation clicker, remote control, etc.).

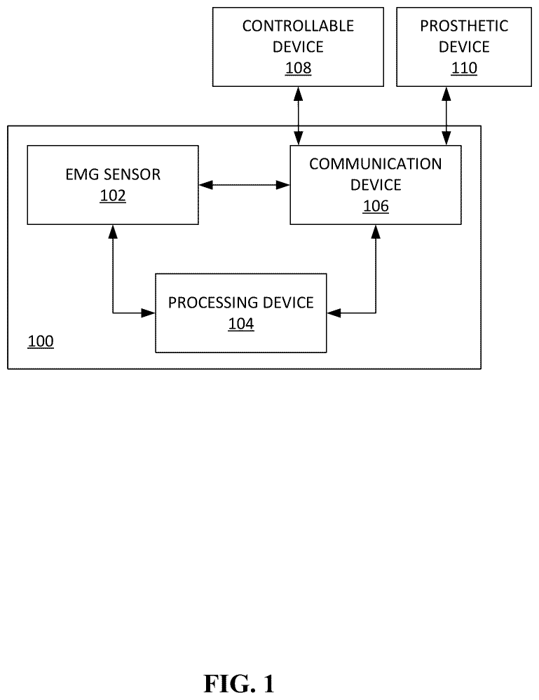

FIG. 1 is a block diagram of a system 100 for facilitating controlling of a device, in accordance with some embodiments. Accordingly, the system 100 may include an Electromyographic (EMG) sensor 102 configured to be attached to one or more of a residual limb of a user and a part of a body of the user. Further, the part of the body may be proximal to a limb of the user. Further, the EMG sensor 102 may be configured to generate an EMG data based on an activity of a muscle of the user. Further, one or more of the residual limb and the part may include the muscle. Further, the system 100 may include a processing device 104 communicatively coupled with the EMG sensor 102. Further, the processing device 104 may be configured to analyze the EMG data. Further, the processing device 104 may be configured to determine an EMG pattern associated with the EMG data based on the analysis. Further, the EMG pattern corresponds to an intended action of the user. Further, the processing device 104 may be configured to generate an action data based on the EMG pattern. Further, the action data may include an indication of the intended action of the user. Further, the system 100 may include a communication device 106 communicatively coupled with the processing device 104. Further, the communication device 106 may be configured to transmit the action data to at least one of a controllable device 108 and a prosthetic device 110.

Further, in some embodiments, the prosthetic device 110 may include a prosthetic hand 200.

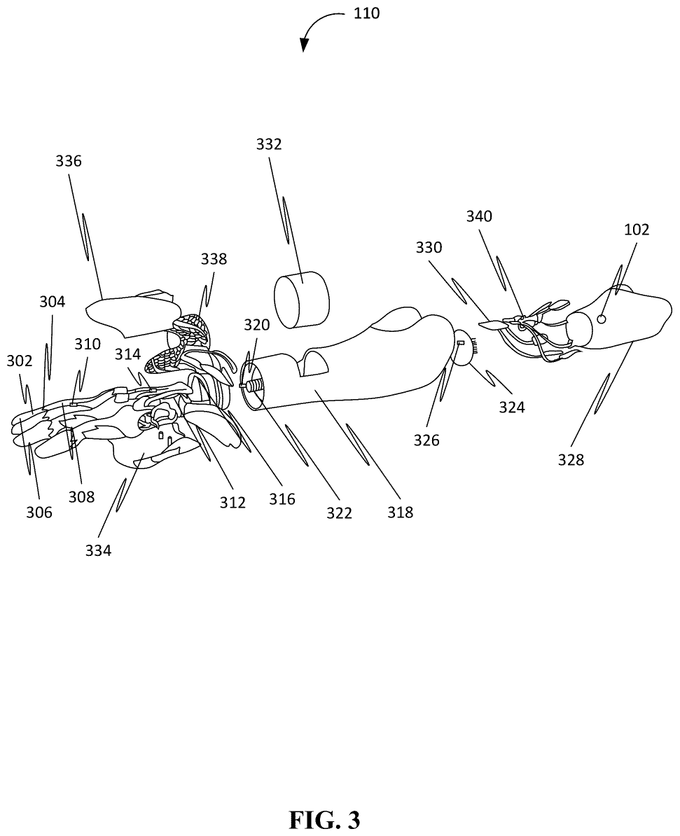

Further, in some embodiments, the prosthetic device 110 may include an artificial finger 302 (as shown in FIG. 3) comprising a joint 304 (as shown in FIG. 3) and a plurality of segments 306-308 (as shown in FIG. 3). Further, the joint 304 may be configured to couple a first segment of the artificial finger 302 to a second segment of the artificial finger 302. Further, the joint 304 may be configured to allow a finger segment movement of the first segment in relation to the second segment. Further, the prosthetic device 110 may include an actuator 310 (as shown in FIG. 3) operationally coupled to each of the first segment and the second segment. Further, the actuator 310 may be configured to cause the finger segment movement.

Further, in some embodiments, the prosthetic device 110 may include a palm assembly 312 (as shown in FIG. 3) and the artificial finger 302. Further, the palm assembly 312 may be operationally coupled to the artificial finger 302. Further, the coupling allows a palm movement of the artificial finger 302 in relation to the palm assembly 312. Further, an actuator 314 (as shown in FIG. 3) may be configured to be operationally coupled to each of the palm assembly 312 and the artificial finger 302. Further, the actuator 314 may be configured to cause the palm movement.

Further, in some embodiments, the prosthetic device 110 may include a wrist assembly 316 (as shown in FIG. 3) and the palm assembly 312. Further, the wrist assembly 316 may be operationally coupled to the palm assembly 312. Further, the coupling allows a wrist movement of the palm assembly 312 in relation to at least one of the residual limb of the user and a forearm assembly 318 (as shown in FIG. 3). Further, the prosthetic device 110 may include the forearm assembly 318. Further, the prosthetic device 110 may include an actuator 320 (as shown in FIG. 3) operationally coupled to each of the palm assembly 312 and at least one of the wrist assembly 316, the residual limb of the user and the forearm assembly 318. Further, the actuator 320 may be configured to cause the wrist movement based on the action data.

Further, in some embodiments, the prosthetic device 110 may include a forearm socket assembly 322 (as shown in FIG. 3) and the wrist assembly 316. Further, a first end of the forearm socket assembly 322 may be configured to be coupled with the wrist assembly 316. Further, a second end of the forearm socket assembly 322 may be configured to be attached to the residual limb of the user.

Further, in some embodiments, the prosthetic device 110 may include the forearm assembly 318 and an upper socket assembly 324 (as shown in FIG. 3). Further, the forearm assembly 318 may be operationally coupled with the upper socket assembly 324. Further, the coupling allows a forearm movement of the forearm assembly 318 in relation to the residual limb of the user. Further, an actuator 326 (as shown in FIG. 3) may be configured to be operationally coupled with the forearm assembly 318 and the upper socket assembly 324. Further, the actuator 326 may be configured to cause the forearm movement based on the action data.

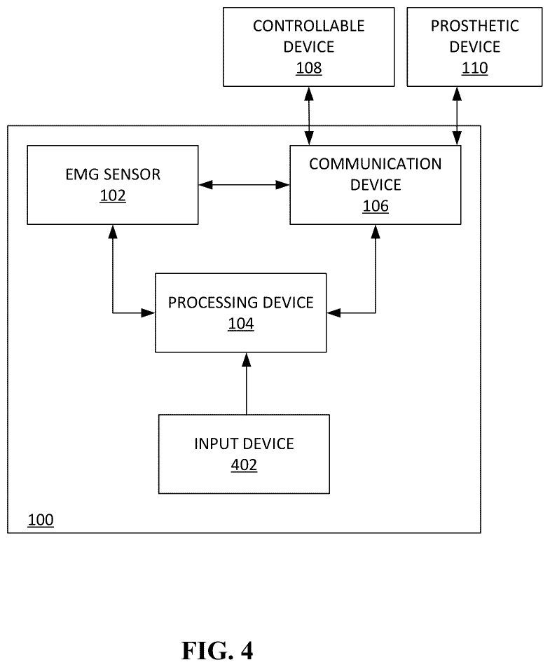

In further embodiments, the system 100 may include an input device 402 (as shown in FIG. 4) configured to receive a toggling input. Further, the toggling input may be associated with an indication of selection of a mode from a plurality of modes. Further, the plurality of modes may include a first mode in which the communication device 106 may be configured to transmit the action data to the controllable device 108. Further, the plurality of modes may include a second mode in which the communication device 106 may be configured to transmit the action data to the prosthetic device 110. Further, the plurality of modes may include a third mode in which the communication device 106 may be configured to transmit the action data to the controllable device 108 and the prosthetic device 110.

Further, in some embodiments, the action data corresponds to at least one of an application function performed by an external application executed on the controllable device 108 and a device function provided by the controllable device 108.

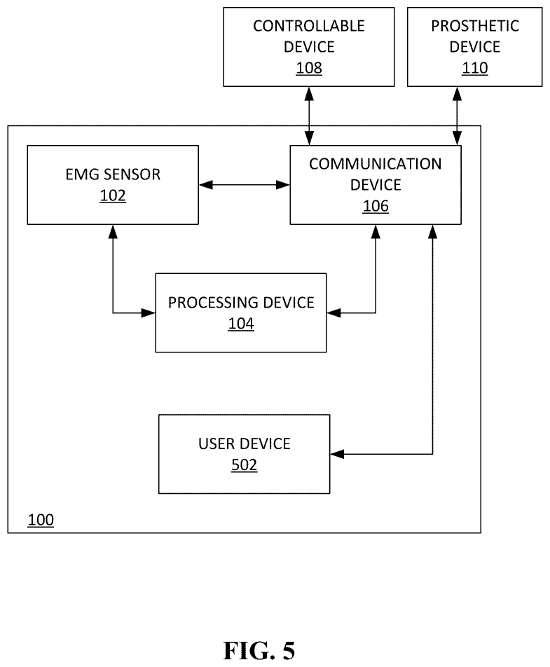

Further, in some embodiments, the communication device 106 may be configured to communicate with a user device 502 (as shown in FIG. 5) that may include a user processing device configured to execute instructions corresponding to a management application. Further, the user device 502 may be configured to present a user interface configured to receive an indication of an association between the action data and at least one of the application function and the device function.

Further, in some embodiments, the analysis of the EMG data may be performed using a machine learning model.

Further, in some embodiments, the controllable device 108 may include one or more of a gamepad and a VR device.

Further, in some embodiments, the controllable device 108 may be a computer device. Further, at least one of a motion and an action of a pointer in a graphical user interface of the computer device may be controlled based on the action data.

Further, in some embodiments, the communication device 106 may be a Bluetooth module.

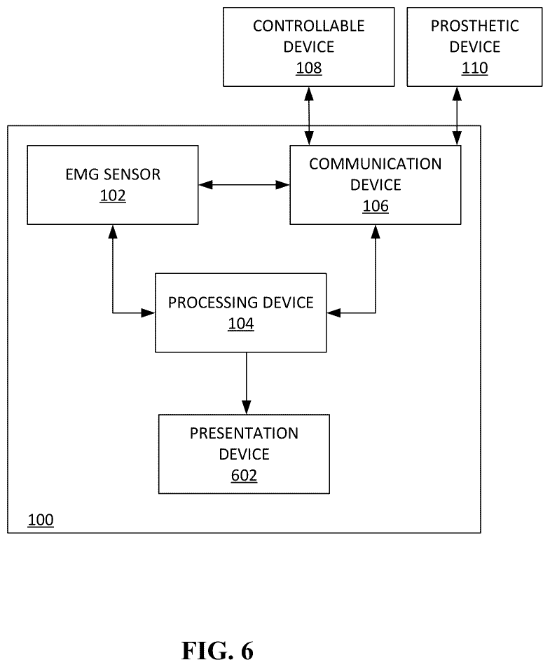

Further, in some embodiments, the communication device 106 may be configured to receive a feedback data from the controllable device 108. Further, the feedback data may be associated with a performance of the intended action by the controllable device 108. Further, the system 100 may include a presentation device 602 (as shown in FIG. 6) configured to present the feedback data.

Further, in some embodiments, the presentation device 602 may include a haptic actuator configured to generate a tactile stimulus based on the feedback data. Further, the tactile stimulus may be perceivable by the user.

Further, in some embodiments, the presentation device 602 may include a light source configured to generate a visual signal based on the feedback data. Further, the visual signal may be perceivable by the user.

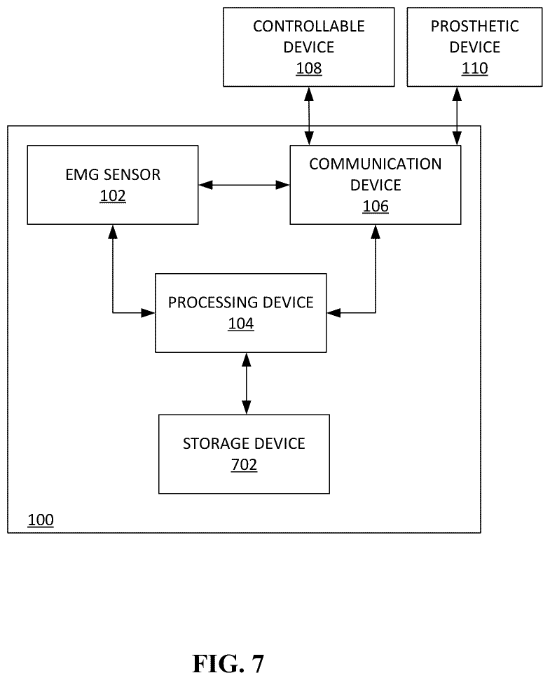

In further embodiments, the system 100 may include a storage device 702 (as shown in FIG. 7) communicatively coupled with the processing device 104. Further, the storage device 702 may be configured to store one or more of the action data and the EMG data.

Further, in some embodiments, the prosthetic device 110 may include a liner 328 (as shown in FIG. 3) configured to be disposed over a portion of the residual limb. Further, the liner 328 may include the EMG sensor 102. Further, in some embodiments, the prosthetic device 110 may include a removable rechargeable power source 332. Further, in some embodiments, the prosthetic device 110 may include a bottom cover 334. Further, in some embodiments, the prosthetic device 110 may include a top cover 336. Further, in some embodiments, the prosthetic device 110 may include a waterproof silicon enclosure 338. Further, in some embodiments, the prosthetic device 110 may include an EMI shielded FFC 340. Further, the liner 328 may be a skin safe breathable liner with shock absorbing pattern.

Further, in some embodiments, the EMG sensor 102 may include a sensor board 330 (as shown in FIG. 3), a sensor patch, and a connector cable. Further, the EMG data may be based on an electrical signal generated by the muscle of the user associated with the sensor patch. Further, the electrical signal may be based on the activity of the muscle of the user. Further, the connector cable may be configured to transfer the electrical signal from the sensor patch to the sensor board 330 configured to generate the EMG data based on the electrical signal.

Further, in some embodiments, the EMG sensor 102 may include a plurality of sensor patches configured to attach to the residual limb of the user.

Further, in some embodiments, the connector cable may include an EMI-shielded flexible flat cable.

Further, in some embodiments, the electrical signal may be generated by the muscle of the user based on at least one of a contraction action of the muscle and a relaxation action of the muscle.

Further, in some embodiments, the EMG sensor 102 may be configured to attach to a surface of a skin associated with at least one of the residual limb and the part of the body of the user. Further, the residual limb and the part comprise the muscle.

Further, in some embodiments, the EMG sensor 102 may include a penetrating needle configured to attach to the user. Further, the penetrating needle detects the electrical signal generated by the muscle of the user.

Further, in some embodiments, the processing device 104 may include an electronic device configured to execute a set of instructions.

Further, in some embodiments, the EMG pattern may include a periodic change in the electrical signal generated by the muscle of the user based on the intended action.

In further embodiments, the system 100 may include an accelerometer sensor configured to generate an accelerometer data. Further, the processing device 104 may be configured to analyze the accelerometer data to determine a movement of the system 100 in a 3-dimensional space.

In further embodiments, the system 100 may include a gyroscope sensor configured to generate a gyroscope data, where the processing device 104 may be configured to analyze the gyroscope data to determine an orientation of the system 100 in the 3-dimensional space.

Further, in some embodiments, the intended action of the user may include a movement executed to perform a task.

Further, in some embodiments, the communication device 106 may be an electronic device configured to at least one of transmit and receive a data.

Further, in some embodiments, the at least one of transmit and receive the data may be performed wirelessly.

Further, in some embodiments, the prosthetic device 110 may include an artificial device configured to at least one of replace a missing limb of the user and improve a functionality of an existing limb of the user.

Further, in some embodiments, the prosthetic device 110 may include a prosthetic leg.

Further, in some embodiments, the prosthetic device 110 may include an artificial foot and a pylon. Further, the artificial foot may be operatively coupled with the pylon. Further, the coupling allows a foot movement of the artificial foot in relation to the pylon. Further, the system 100 may include an actuator configured to operatively coupled with the artificial foot and the pylon. Further, the actuator may be configured to cause the foot movement.

Further, in some embodiments, the prosthetic device 110 may include the knee socket and a pylon. Further, the knee socket may be operatively coupled with the pylon. Further, a first end of the knee socket may be attached to the residual limb of the user and the second end of the knee socket may be attached to the pylon. Further, the coupling allows a knee movement of the pylon in relation to the residual limb of the user. Further, an actuator may be configured to operatively coupled with the knee socket and the pylon. Further, the actuator may be configured to cause the knee movement.

Further, in some embodiments, the EMG sensor 102 may be disposed over the knee socket.

Further, in some embodiments, the artificial finger 302 may be configured to at least one of mimic a functionality of a finger of the user and assist an existing finger of the user.

Further, in some embodiments, the finger segment movement may include mimicking at least one of a flexion movement, an extension movement, an adduction movement, and an abduction movement of a finger of the user.

Further, in some embodiments, the actuator may include one or more of a motor, an electrically controlled muscle, and a fluid-based hydraulic piston.

Further, in some embodiments, the actuator may be coupled with the artificial finger 302 using an artificial tendon configured to convert the mechanical energy to one or more of a translational motion and an angular motion.

In further embodiments, the prosthetic device 110 may include a waterproof silicone enclosure configured to enclose the palm assembly 312. Further, the waterproof silicone enclosure may be configured to protect the palm assembly 312 from moisture.

In further embodiments, the prosthetic device 110 may include at least one of a top cover and a bottom cover configured to house the palm assembly 312.

Further, in some embodiments, the EMG sensor 102 may be disposed over the forearm socket assembly 322.

Further, in some embodiments, the motor may include an electrical device configured to provide rotational motion.

Further, in some embodiments, the hydraulic system may be a fluid-based hydraulic piston.

Further, in some embodiments, the EMG sensor 102 may be disposed over the upper socket assembly 324.

Further, in some embodiments, the forearm assembly 318 may include an energy-storing device configured to supply power to the EMG sensor 102, the motor, the communication device 106, and the processing device 104.

Further, in some embodiments, the energy storing device may include an energy management circuit electrically coupled with the energy storing device. Further, the energy management circuit may be configured to perform at least one of monitor the energy-storing device, protect the energy-storing device from one or more of an overcharge of the energy-storing device, and estimate a state of the energy-storing device.

Further, in some embodiments, the energy-storing device may be configured to be detachable from the forearm assembly 318.

Further, in some embodiments, the energy storing device may be configured to be round shaped.

Further, in some embodiments, the liner 328 may include a shock absorbing pattern.

Further, the shock absorbing pattern may include storing mechanical energy from an impact to the prosthetic hand 200. Further, the storing the mechanical energy facilitates reduction in transfer of the mechanical energy from the impact to the residual limb of the user.

Further, in some embodiments, the input device 402 may include at least one of a button, a lever, a dial, and a touchpad.

Further, in some embodiments, the communication device 106 may be configured to communicate with the user device 502 comprising a user processing device configured to execute instructions corresponding to a management application. Further, the user device 502 may be configured to present a user interface configured to receive a training instruction associated with training of the machine learning model.

Further, in some embodiments, a machine learning model may include a software application configured to recognize a pattern in a dataset to perform one or more of a decision-making action and prediction actions on a new dataset.

Further, in some embodiments, the machine learning model may include a machine learning algorithm and the dataset.

Further, in some embodiments, the machine learning model may include one or more of a supervised learning model, an unsupervised learning model, and a reinforcement learning model.

Further, in some embodiments, the gamepad may include a piece of equipment may include at least one of a user input device and a plurality of gamepad sensors. Further, the gamepad may be configured to at least one of interact with a computer device and provide an input to the computer device based on at least one of a user input received from the user input device and the plurality of gamepad sensors.

Further, in some embodiments, the VR device may include a virtual reality device comprising a virtual hand configured to be controlled based on the action data.

Further, in some embodiments, the Bluetooth module may include an electronic circuit configured to implement Bluetooth technology configured to facilitate wireless data transmission.

Further, in some embodiments, the storage device 702 may include a non-volatile memory.

Further, in some embodiments, the wrist movement may include mimicking at least one of a flexion movement, an extension movement, an adduction movement, and an abduction movement of a wrist of the user.

In further embodiments, the system 100 may include the input device 402 configured to receive a toggling input. Further, the toggling input may be associated with an indication of selection of a mode from a plurality of modes. Further, the plurality of modes may include a VR mode in which the communication device 106 may be configured to transmit the action data to a VR device may include a virtual hand configured to be controlled by the action data.

Further, the plurality of modes may include a mouse mode in which the communication device 106 may be configured to transmit the action data to a computer mouse causing one or more of a movement and clicking actions of a pointer in a graphical user interface of a computing device associated with the computer mouse. Further, the plurality of modes may include a gamepad mode in which the communication device 106 may be configured to transmit the action data to a gamepad may include a trigger. Further, actuation of the trigger may be based on the action data. Further, the plurality of modes may include a prosthetic mode in which the communication device 106 may be configured to transmit the action data to the prosthetic device 110.

Further, in some embodiments, the communication device 106 may be further configured to communication with a user device comprising a user processing device, a user presentation device and a user communication device. Further, the user device may be configured to execute instructions associated with a management application. Further, the management application may be configure to present a plurality of user profiles comprising a first user profile corresponding to a first functioning characteristics of the prosthetic device. Further, the plurality of user profiles may include a second user profile corresponding to a second functioning characteristics of the prosthetic device. Further, the user device may be configured to receive a selection data associated with indication of selection of a user profile from a plurality of user profiles. Further, the communication device 106 may be configured to receive the user profile corresponding to the selection data. Further, the generation of the action data may be based on the user profile.

Further, in some embodiments, the system 100 may include an energy management device comprising an energy storage device and an energy management circuit. Further, the energy management device may be coupled with the processing device. Further, the energy management circuit may be configured to determine one or more of a energy storage device health parameter, a charge status associated with the energy storage device and a power consumption associated with the prosthetic device.

Further, in some embodiments, the communication device 106 may be configured to communicate with a user device. Further, the analysis of EMG data may be performed using a pre-trained machine learning model received from the user device. Further, the user device may be configured to train the pre-trained machine learning model based on the EMG data received from the communication device 106.

In further embodiments, disclosed herein is a method of facilitating controlling of a device. Accordingly, the method may include receiving, using a communication device, an EMG data from a system 100 that may include an Electromyographic (EMG) sensor configured to generate the EMG data based on an activity of a muscle of a user. Further, the EMG sensor 102 may be configured to be attached to one or more of a residual limb of the user and a part of a body of the user. Further, the part of the body may be proximal to a limb of the user. Further, at least one of the residual limb and the part may include the muscle. Further, the method may include analyzing, using a processing device, the EMG data. Further, the method may include determining, using the processing device, an EMG pattern associated with the EMG data based on the analysis. Further, the EMG pattern corresponds to an intended action of the user. Further, the method may include generating, using the processing device, an action data based on the EMG pattern. Further, the action data may include indication of the intended action of the user. Further, the method may include transmitting, using a communication device, the action data to the system 100 configured to transmit the action data, using a system communication device, to at least one of the controllable device 108 and the prosthetic device 110.

Further, in some embodiments, the method may include storing, using a storage device, one or more of the action data and the EMG data.

Further, in some embodiments, the action data corresponds to at least one of an application function performed by an external application executed on the controllable device 108 and a device function provided by the controllable device 108.

Further, in some embodiments, the method may include receiving, using the communication device, an indication of an association between the action data and at least one of the application function and the device function. Further, the indication of the association may be received from the user device 502 comprising a user processing device configured to execute instructions corresponding to a management application. Further, the user device 502 may be configured to present a user interface configured to receive the indication of the association.

Further, in some embodiments, the analysis of the EMG data may be performed using a machine learning model.

In further embodiments, disclosed herein is a method of facilitating training of a machine learning model configured for analysing EMG data. Accordingly, the method may include receiving, using a communication device, a training data set may include a plurality of EMG patterns corresponding to a plurality of intended actions. Further, the method may include training, using a processing device, the machine learning model based on the training data set.

In further embodiments, the method may include receiving, using the communication device, a plurality of EMG data corresponding to a plurality of gestures. Further, the plurality of EMG data may be received from the system 100 that may include an EMG sensor 102 configured to generated the plurality of EMG data. Further, the method may include analysing, using the processing device, the plurality of EMG data. Further, the method may include determining, using the processing device, at least one feature of an EMG data of the plurality of EMG data. Further, the at least one feature corresponds to each gesture. Further, the at least one feature may be associated with an EMG pattern. Further, the method may include generating the training data set based on determining the at least one feature.

Further, in some embodiments, the method may include receiving, using a communication device, a training instruction associated with training of the machine learning model. Further, the training instruction may be received from the user device 502 comprising a user processing device configured to execute instructions corresponding to a management application. Further, the user device 502 may be configured to present a user interface configured to receive the training instruction.

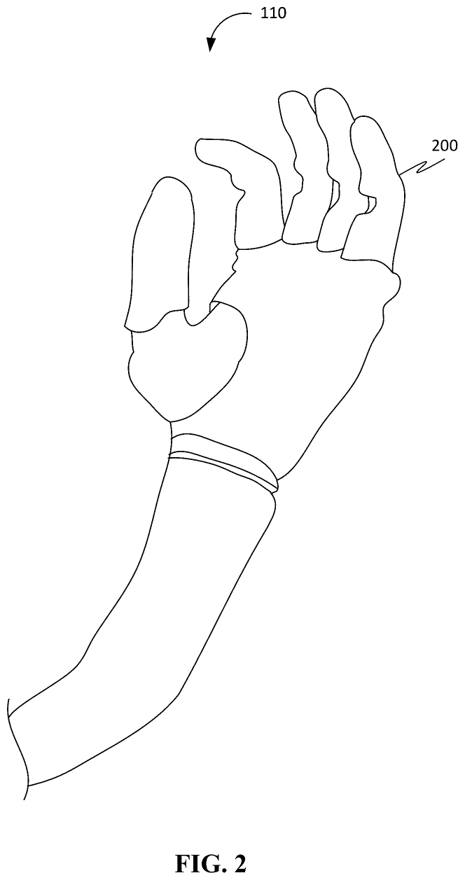

FIG. 2 illustrates the prosthetic device 110 for facilitating controlling of the device, in accordance with some embodiments.

FIG. 3 is an exploded view of the prosthetic device 110 for facilitating controlling of the device, in accordance with some embodiments.

FIG. 4 is a block diagram of the system 100 for facilitating controlling of the device, in accordance with some embodiments.

FIG. 5 is a block diagram of the system 100 for facilitating controlling of the device, in accordance with some embodiments.

FIG. 6 is a block diagram of the system 100 for facilitating controlling of the device, in accordance with some embodiments.

FIG. 7 is a block diagram of the system 100 for facilitating controlling of the device, in accordance with some embodiments.

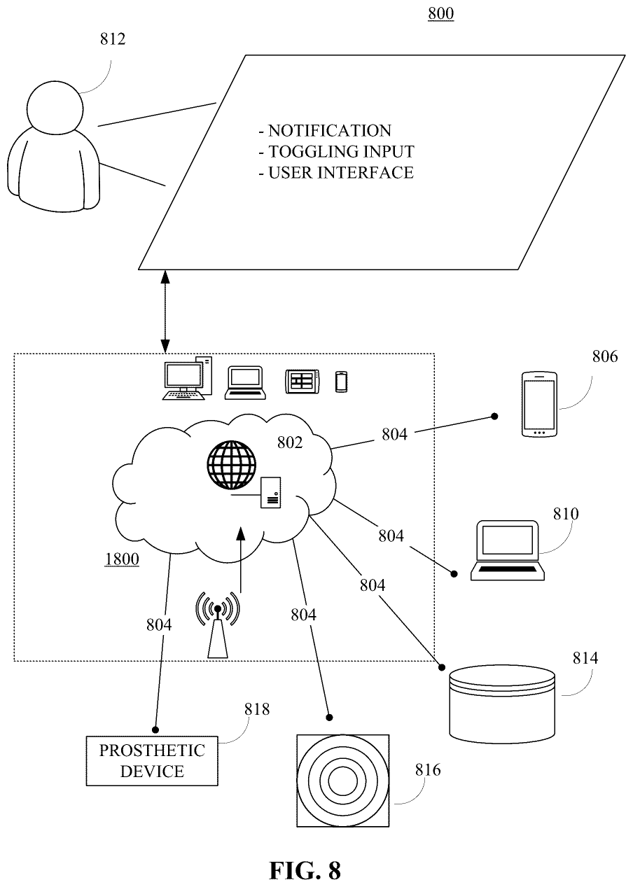

FIG. 8 is an illustration of an online platform 800 consistent with various embodiments of the present disclosure. By way of non-limiting example, the online platform 800 to facilitate controlling a device may be hosted on a centralized server 802, such as, for example, a cloud computing service. The centralized server 802 may communicate with other network entities, such as, for example, a mobile device 806 (such as a smartphone, a laptop, a tablet computer, etc.), other electronic devices 810 (such as desktop computers, server computers, etc.), databases 814, sensors 816, device 818 (such as the prosthetic device 110) over a communication network 804, such as, but not limited to, the Internet. Further, users of the online platform 800 may include relevant parties such as, but not limited to, end-users, administrators, service providers, service consumers, and so on. Accordingly, in some instances, electronic devices operated by the one or more relevant parties may be in communication with the platform.

A user 812, such as the one or more relevant parties, may access online platform 800 through a web based software application or browser. The web based software application may be embodied as, for example, but not be limited to, a website, a web application, a desktop application, and a mobile application compatible with a computing device 1800.

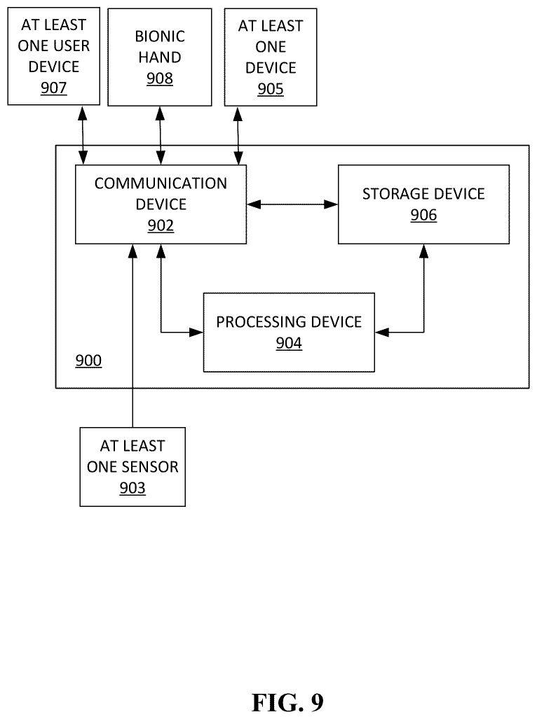

FIG. 9 is a block diagram of a system 900 for facilitating controlling devices based on gesture recognition using a bionic hand 908, in accordance with some embodiments. Accordingly, the system may include a communication device 902 configured for receiving at least one sensor data from at least one sensor 903. Further, the at least one sensor 903 may include at least one EMG sensor, a motion sensor, an accelerometer, a gyroscope, an orientation sensor, etc. Further, the at least one sensor 903 may be configured for generating the at least one sensor data based on an action or gesture performed by at least one user. Further, the at least one user may include an individual that may want to control the devices using the bionic hand 908. Further, the communication device 902 may be configured for transmitting a gesture information to the bionic hand 908. Further, the bionic hand 908 may include at least one actuator mechanically coupled with at least one of at least one prosthetic finger and a wrist joint comprised in the bionic hand 908. Further, the at least one actuator may include a DC motor with a specific gear setup. Further, the at least one actuator may be configured for moving the at least one of at least one prosthetic finger and the wrist joint based on the gesture information. Further, the communication device 902 may be configured for transmitting a command to at least one device 905. Further, the at least one device 905 may include at least one target Bluetooth device such as a mouse or a gamepad. Further, the communication device 902 may be configured for transmitting a notification to at least one user device 907 associated with the at least one user. Further, the notification may indicate that an action corresponding to the command has been performed. Further, the at least one user device 907 may include a smartphone, a laptop, a mobile, a personal computer, etc. Further, the notification may include a sound notification, a vibration notification, and a light notification.

Further, the system 900 may include a processing device 904 configured for analyzing the at least one sensor data using at least one machine learning model, wherein the at least one machine learning model may be based on at least one machine learning algorithm. Further, the processing device 904 may be configured for determining the gesture information associated with a gesture based on the analyzing. Further, the processing device 904 may be configured for generating a command based on the determining of the gesture. Further, the command may correspond to the action that the at least one user may want to perform based on the gesture.

Further, the system 900 may include a storage device 906 communicatively coupled with the communication device. Further, the storage device may be configured for storing the at least one sensor data, the gesture information, the command, and the notification.

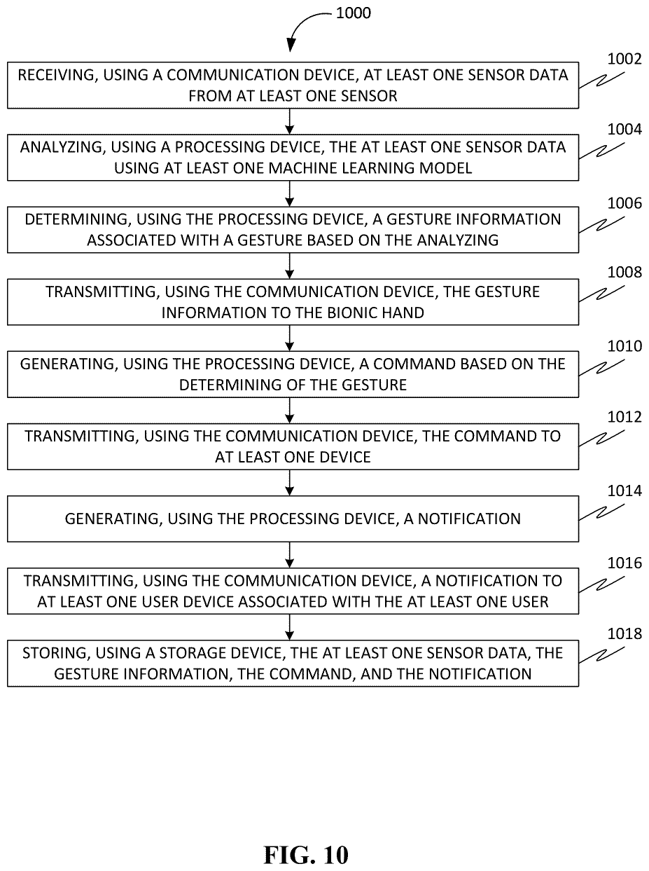

FIG. 10 is a flow chart of a method 1000 for facilitating controlling devices based on gesture recognition using a bionic hand, in accordance with some embodiments. Accordingly, at 1002, the method 1000 may include receiving, using a communication device, at least one sensor data from at least one sensor. Further, the at least one sensor may include at least one EMG sensor, a motion sensor, an accelerometer, a gyroscope, an orientation sensor, etc. Further, the at least one sensor may be configured for generating the at least one sensor data based on an action or gesture performed by at least one user. Further, the at least one user may include an individual that may want to control the devices using the bionic hand. Further, in some embodiments, the at least one sensor may be comprised in the bionic hand.

Further, at 1004, the method 1000 may include analyzing, using a processing device, the at least one sensor data using at least one machine learning model, wherein the at least one machine learning model may be based on at least one machine learning algorithm.

Further, at 1006, the method 1000 may include determining, using the processing device, a gesture information associated with a gesture based on the analyzing.

Further, at 1008, the method 1000 may include transmitting, using the communication device, the gesture information to the bionic hand. Further, the bionic hand may include at least one actuator mechanically coupled with at least one prosthetic finger and a wrist joint comprised in the bionic hand. Further, the at least one actuator may be configured for moving at least one of at least one prosthetic finger and the wrist joint based on the gesture information.

Further, at 1010, the method 1000 may include generating, using the processing device, a command based on the determining of the gesture. Further, the command may correspond to the action that the at least one user may want to perform based on the gesture.

Further, at 1012, the method 1000 may include transmitting, using the communication device, the command to at least one device. Further, the at least one device may include at least one target Bluetooth device. Further, the at least one user may use the bionic hand to control the at least one target Bluetooth device. Further, in an instance, the at least one target Bluetooth device may include a personal computer. Further, the at least one user may use the bionic hand as a mouse or a gamepad, a presentation clicker, a remote control, a VR controller, etc. to control the personal computer.

Further, at 1014, the method 1000 may include generating, using the processing device, a notification.

Further, at 1016, the method 1000 may include transmitting, using the communication device, a notification to at least one user device associated with the at least one user. Further, the notification may indicate that an action corresponding to the command has been performed. Further, the at least one user device may include a smartphone, a laptop, a mobile, a personal computer, etc.

Further, at 1018, the method 1000 may include storing, using a storage device, the at least one sensor data, the gesture information, the command, and the notification.

Further, in some embodiments, the prosthetic device 110 may include the bionic hand that may include a prosthetic hand comprising a physical artificial hand with motorized fingers and a wrist joint. Further, the bionic hand may include a communicator (such as the communication device), a processor (such as the processing device), and a data storage device (such as the storage device). Further, the bionic hand may include eight EMG sensors strategically placed on the user's limb or muscles, wherein the EMG sensors detect these patterns, convert them into digital signals, and transmit the information to the bionic hand's control system. Accordingly, the bionic hand may include a replaceable battery with built in BMS, a female wrist assembly, a male wrist assembly, and a waterproof silicone enclosure. Further, the replaceable battery may be characterized by a round shape. Further, the bionic hand may include a top cover and a bottom cover. Further, the bionic hand may include a mechanical palm assembly. Further, the bionic hand may include a control board, a sensor board, an EMI-shielded FFC, and an octusense. Further, the bionic hand may include a skin-safe breathable liner with a shock-absorbing pattern.

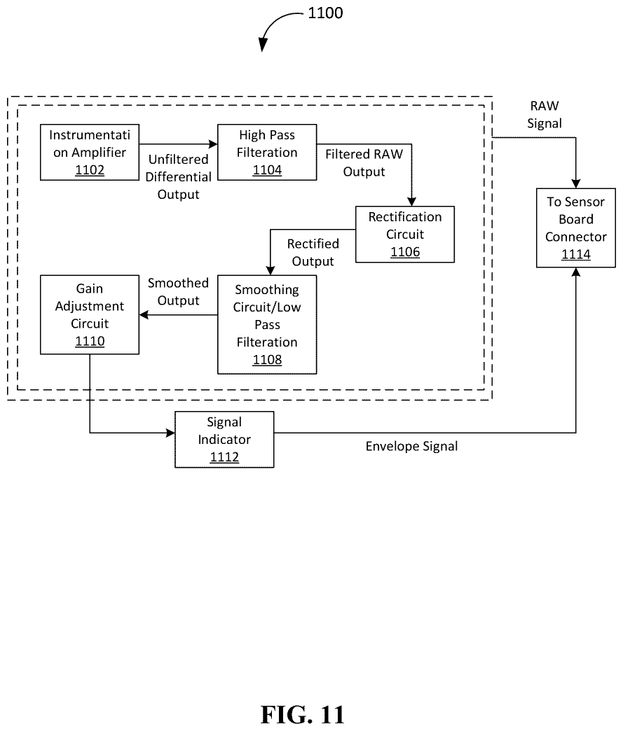

FIG. 11 illustrates an EMG electrode board 1100 comprised in a system for facilitating controlling the devices based on gesture recognition, in accordance with some embodiments. Accordingly, the system may include the system 100. Further, the EMG electrode board 1100 may be used to sense voltages generated by the muscles, amplify the voltages, and process the signal to generate several waveforms that may then be used to extract features for the machine learning models. FIG. 11 illustrates how the signal is acquired.

Further, at 1114, the EMG electrode board 1100 may include a sensor board connector. Further, at 1102, the EMG electrode board 1100 may include an instrumentation amplifier that may provide unfiltered differential output. Further, at 1104, the EMG electrode board 1100 may perform high-pass filtration. Further, at 1106, the EMG electrode board 1100 may provide a filtered RAW output to a rectification circuit that may provide a rectified output. Further, at 1108, the EMG electrode board 1100 may include a smoothing circuit that may perform low pass filtration. Further, at 1110, the EMG electrode board 1100 may include a gain adjustment circuit that may receive a smoothed output from the smoothing circuit. Further, at 1112, the EMG electrode board 1100 may include a signal indicator that may send an envelope signal to the sensor board connector.

The EMG electrode board 1100 may include 3 dry contact pads connected to a bottom layer. The pads represent a middle muscle contact area, an end muscle contact area, and a reference muscle for the signal to base upon.

The EMG electrode board 1100 may have 5 stages of signal acquisition.

Further, stage 1 may include differential Signal detection and amplification. Further, in this stage, an instrumentation amplifier may be used to amplify the difference in voltage generated by the end muscle versus the middle muscle. The values are references based on the reference muscle signal. Further, the pads may be directly connected to the negative and positive inputs of the amplifier. Further, the pad may be directly connected to the reference pin within the amplifier. At this stage, the signal is amplified more than 200×. The output signal of the first stage is an unfiltered RAW signal that contains a lot of noise.

Further, stage 2 may include high-pass filtration. Further, an inverting operational amplifier circuit may be used as an active high-pass filter to eliminate the low-frequency signals, therefore, reducing the noise. The gain Av is intended to be −1 and the cutoff frequency is intended to be around 106 Hz. At this stage, the output RAW signal is stabilized and the noise within is reduced.

Further, stage 3 may include signal rectification. Further, an active full wave rectification circuit is used to convert the negative signal to a positive signal. Active rectification may be used to ensure lower losses within the circuit and to prevent output saturation.

Further, stage 4 may include signal smoothing. Within this stage, the rectified signal may be used as the input of a simplified inverting amplifier filter circuit. The gain Av of this circuit is −1. Since this circuit operates as a low pass filter also, the cutoff frequency is 2 Hz.

Further, stage 5 may include gain amplification. Further, this stage is used to amplify the smooth signal acquired further and as desired. This gain could be implemented as much as the user desires.

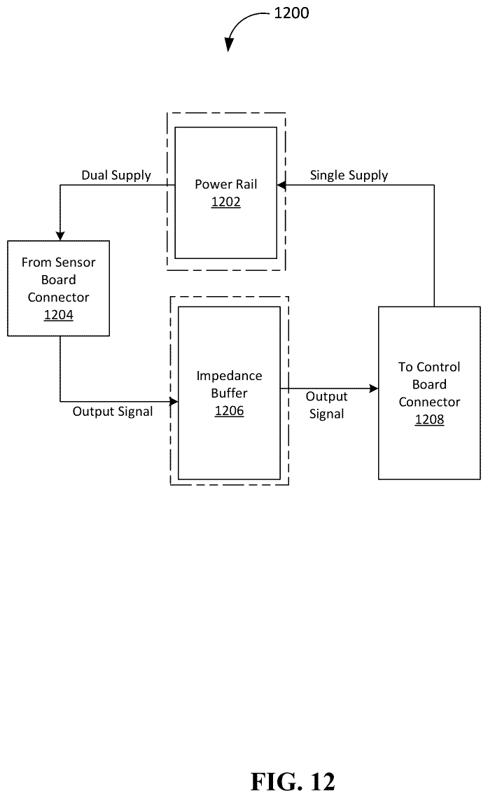

FIG. 12 illustrates a sensor board 1200 of the system for facilitating controlling the devices based on gesture recognition, in accordance with some embodiments. Accordingly, the sensor board 1200 may be used to supply the electrode board with positive and negative voltages. Further, the sensor board 1200 may include an impedance buffer 1206 to ensure low resistance over the connection from the electrode board to a control board. Therefore, the signal may be transmitted smoothly. Further, a charge pump may be used in the process of generating a dual supply source. Further, an operational amplifier may be used to create the impedance buffer circuit. Further, the sensor board may combine the signal from all the EMG sensors and provide them with electricity while sending the data received to the control board. Further, the sensor board 1200 may include a power rail 1202 that may provide a dual supply to a sensor board connector 1204 that may provide an output signal to the impedance buffer 1206. Further, the impedance buffer 1206 may provide the output signal to a control board connector 1208.

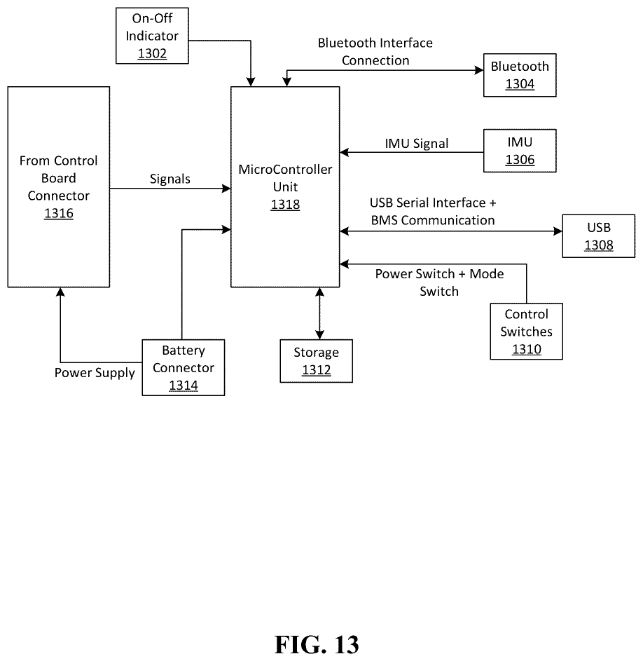

FIG. 13 is a block diagram of a main board 1300 for facilitating controlling the devices based on gesture recognition, in accordance with some embodiments. Accordingly, the main board 1300 (or control board) may include a microcontroller unit 1318 (or microcontroller) that is powerful enough to operate several tasks in parallel. FIG. 13 illustrates components of the main board 1318.

Further, various tasks may be operated by the microcontroller unit 1318.

Further, at 1310, the control board 1300 may include two switches. Further, a first switch of the two switches may be used for on-off toggling and a second switch of the two switches may be used for mode toggling.

Further, once the control board 1300 is connected to the supply and the toggle switch is pressed an indicator light 1302 is turned on and once the switch is repressed the board and indicator light 1302 are turned off.

Further, a USB connector 1308 may be used to communicate with a serial device (PC or laptop) and may be used to configure a battery management system when charging the battery.

Further, the control board 1300 may include a Bluetooth interface 1304 equipped within the control board 1300 for a user interface (mobile app and Windows app).

Further, the control board may include a battery connector 1314 used to power the sensor board and the electrode boards.

Further, an IMU 1306 may send a signal to the microcontroller unit 1318 to configure an axis orientation of the control board 1300 and this signal may be used in the machine learning process.

Further, the control board 1300 may be configured for storing a data since the control board 1300 may be provided with the ability to store data for the machine learning models in a storage 1312.

The microcontroller unit 1318 may control all of the tasks above in addition to processing incoming signals and analyzing the required movement. Further, the control board 1300 may include a control board connector 1316.

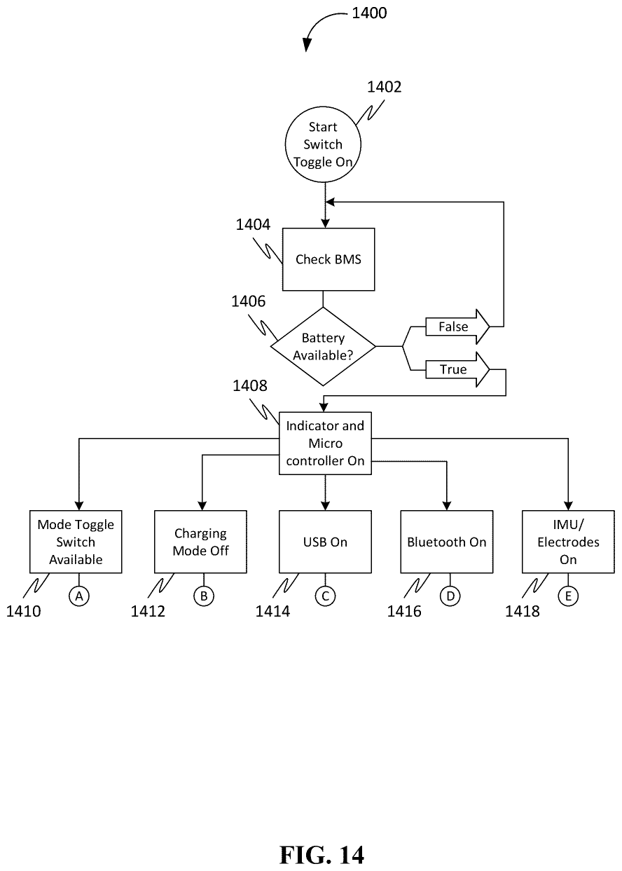

FIG. 14 is a flow diagram of a method 1400 for facilitating controlling the devices based on gesture recognition using the bionic hand, in accordance with some embodiments. Accordingly, the method 1400 may include a start switch 1402 that may be toggled on. Further, at 1404, the method 1400 may include checking BMS. Further, at 1406, the method 1400 may include checking if a battery is available. Further, if the battery is not available, the method may proceed to 1404. Further, if the battery is available, at 1408, the method 1400 may proceed to turning an indicator and a micro controller on. Further, at 1410, the microcontroller may be associated with a mode toggle switch. Further, at 1420, the method may include setting hand mode as default on start. Further, at 1422, the method may include checking if the toggle switch is pressed. Further, if the toggle switch is pressed, at 1426, the method may include toggling the mode. Further, if the toggle switch is not pressed, at 1424, the method may include continuing with the current mode. Further, after 1424 and 1426, the method 1400 may proceed to 1428. Further, at 1412, the microcontroller may be associated with turning a charging mode off. Further, after 1412, the method 1400 may proceed to 1428. Further, at 1414, the microcontroller may be associated with a USB. Further, at 1430, the method 1400 may include if the toggle switch is pressed. Further, if the toggle switch is pressed, at 1434, the method may include disabling an operation mode. Further, after 1434, the method 1400 may proceed to 1440. Further, if the toggle switch is not pressed, at 1432, the method may include continuing to the operation mode. Further, at 1416, the microcontroller may be associated with a Bluetooth. Further, after 1416, the method 1400 may proceed to 1440. Further, at 1418, the microcontroller may be associated with IMU/electrodes. Further, at 1436, the method 1400 may include calculating a number of electrodes available. Further, at 1438, the method 1400 may include choosing from a storage intended machine learning model. Further, after 1438, the method 1400 may proceed to 1428. Further, at 1428, the method 1400 may include performing the operation mode. Further, at 1442, the operation mode may be a VR mode. Further, at 1452, the method 1400 may include getting a preset data function VR mode from a storage. Further, at 1460, the method 1400 may include starting VR operation. Further, at 1444, the operation mode may be a game pad mode. Further, at 1454, the method 1400 may include getting preset data functions for the game pad from the storage. Further, at 1462, the method 1400 may include starting a game pad mode. Further, at 1446, the operation mode may be a mouse mode. Further, at 1456, the method 1400 may include getting preset data functions for a mouse from the storage. Further, at 1464, the method may include starting the mouse mode. Further, at 1448, the operation mode may be a hand mode. Further, at 1458, the method 1400 may include getting preset data functions for the hand mode. Further, at 1466, the method may include starting the hand mode. Further, after 1460, 1462, 1464, and 1466, at 1488, the method 1400 may include turning a stop toggle switch off. Further, at 1440, the method 1400 may include performing in a training mode. Further, at 1450, the method 1400 may include turning the Bluetooth interface or serial user interface on. Further, at 1468, the method 1400 may include receiving a signal from electrode boards and IMU. Further, at 1470, the method 1400 may include sending a data to app and saving in a profile record. Further, at 1472, the method 1400 may include processing the signal and creating features for each gesture. Further, at 1474, the method 1400 may include creating a training set and a testing set. Further, at 1476, the method 1400 may include training a model based on data received from each gesture. Further, at 1478, the method 1400 may include loading a specific model under each profile chosen. Further, at 1480, the method 1400 may include testing the model in operation and creating a storage room for the model to be saved upon. Further, at 1482, the method 1400 may include predicting gestures by automatically using data reading and the stored machine learning model in the operation mode. Further, at 1484, the method 1400 may include storing new data upon operation for future calibration. Further, after 1484, the method 1400 may proceed to 1488.

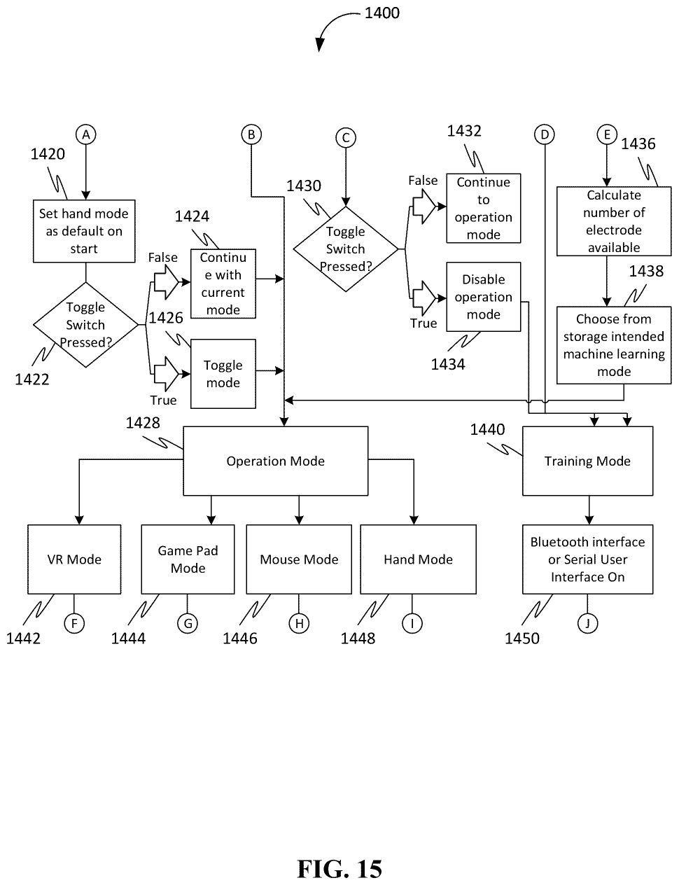

FIG. 15 is a continuation flow diagram of FIG. 14.

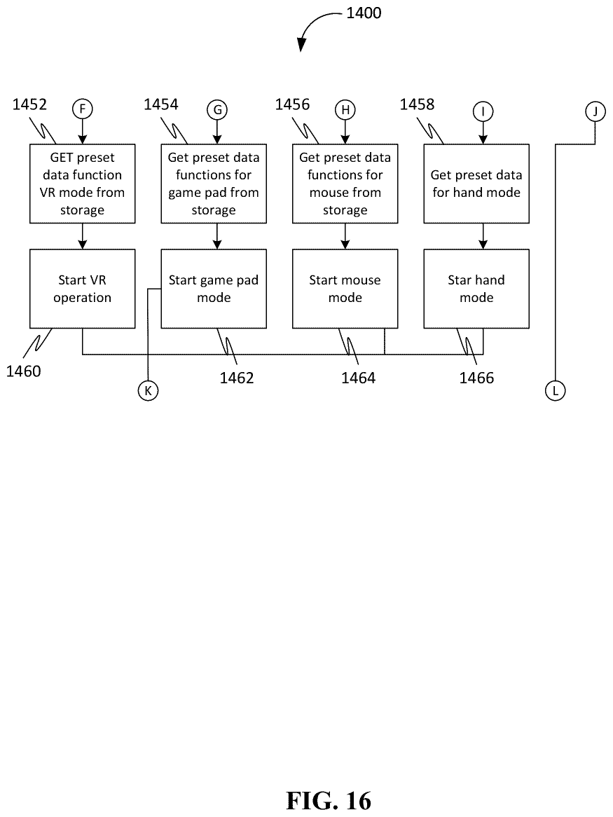

FIG. 16 is a continuation flow diagram of FIG. 14.

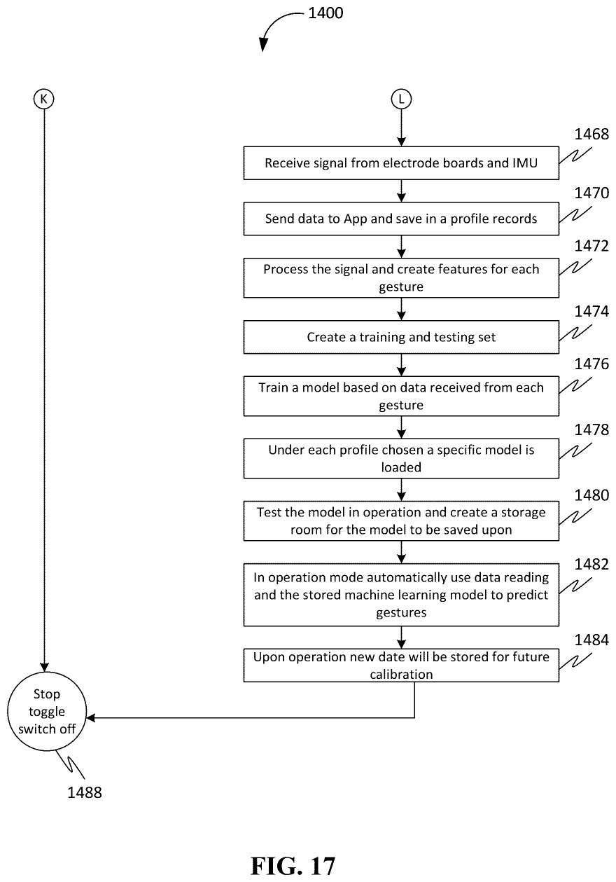

FIG. 17 is a continuation flow diagram of FIG. 14.

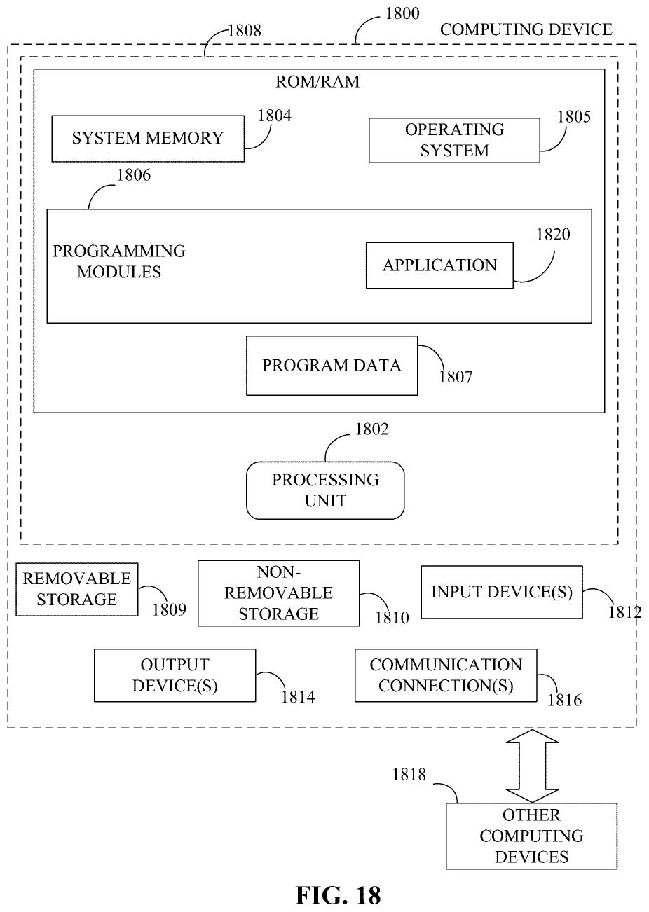

With reference to FIG. 18, a system consistent with an embodiment of the disclosure may include a computing device or cloud service, such as computing device 1800. In a basic configuration, computing device 1800 may include at least one processing unit 1802 and a system memory 1804. Depending on the configuration and type of computing device, system memory 1804 may comprise, but is not limited to, volatile (e.g. random-access memory (RAM)), non-volatile (e.g. read-only memory (ROM)), flash memory, or any combination. System memory 1804 may include operating system 1805, one or more programming modules 1806, and may include a program data 1807. Operating system 1805, for example, may be suitable for controlling computing device 1800's operation. In one embodiment, programming modules 1806 may include image-processing modules, machine learning modules, etc. Furthermore, embodiments of the disclosure may be practiced in conjunction with a graphics library, other operating systems, or any other application program and is not limited to any particular application or system. This basic configuration is illustrated in FIG. 18 by those components within a dashed line 1808.

Computing device 1800 may have additional features or functionality. For example, computing device 1800 may also include additional data storage devices (removable and/or non-removable) such as, for example, magnetic disks, optical disks, or tape. Such additional storage is illustrated in FIG. 18 by a removable storage 1809 and a non-removable storage 1810. Computer storage media may include volatile and non-volatile, removable, and non-removable media implemented in any method or technology for storage of information, such as computer-readable instructions, data structures, program modules, or other data. System memory 1804, removable storage 1809, and non-removable storage 1810 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD), other optical storage, magnetic cassettes, magnetic tape, magnetic disk storage or other magnetic storage devices, or any other medium which can be used to store information and which can be accessed by computing device 1800. Any such computer storage media may be part of device 1800. Computing device 1800 may also have input device(s) 1812 such as a keyboard, a mouse, a pen, a sound input device, a touch input device, a location sensor, a camera, a biometric sensor, etc. Output device(s) 1814 such as a display, speakers, a printer, etc. may also be included. The aforementioned devices are examples and others may be used.

Computing device 1800 may also contain a communication connection 1816 that may allow device 1800 to communicate with other computing devices 1818, such as over a network in a distributed computing environment, for example, an intranet or the Internet. Communication connection 1816 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1804, including operating system 1805. While executing on processing unit 1802, programming modules 1806 (e.g., application 1820 such as a media player) may perform processes including, for example, one or more stages of methods, algorithms, systems, applications, servers, databases as described above. The aforementioned process is an example, and processing unit 1802 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A system for facilitating controlling of a device, the system comprising:
an Electromyographic (EMG) sensor configured to be attached to one or more of a residual limb of a user and a part of a body of the user, wherein the part of the body is proximal to a limb of the user, wherein the EMG sensor is configured to generate an EMG data based on an activity of a muscle of the user, wherein one or more of the residual limb and the part comprises the muscle;
a processing device communicatively coupled with the EMG sensor, wherein the processing device is configured to:
analyze the EMG data;
determine an EMG pattern associated with the EMG data based on the analysis, wherein the EMG pattern corresponds to an intended action of the user; and
generate an action data based on the EMG pattern, wherein the action data comprises an indication of the intended action of the user; and
a communication device communicatively coupled with the processing device, wherein the communication device is configured to transmit the action data to at least one of a controllable device and a prosthetic device.

2. The system of claim 1, wherein the prosthetic device comprises a prosthetic hand.

3. The system of claim 1, wherein the prosthetic device comprises an artificial finger comprising a joint and a plurality of segments, wherein the joint is configured to couple a first segment of the artificial finger to a second segment of the artificial finger, wherein the joint is further configured to allow a finger segment movement of the first segment in relation to the second segment, wherein the prosthetic device comprises an actuator operationally coupled to each of the first segment and the second segment, wherein the actuator is configured to cause the finger segment movement.

4. The system of claim 1, wherein the prosthetic device comprises a palm assembly and an artificial finger, wherein the palm assembly is operationally coupled to the artificial finger, wherein the coupling allows a palm movement of the artificial finger in relation to the palm assembly, wherein an actuator is further configured to be operationally coupled to each of the palm assembly and the artificial finger, wherein the actuator is further configured to cause the palm movement.

5. The system of claim 1, wherein the prosthetic device comprises a wrist assembly and a palm assembly, wherein the wrist assembly is operationally coupled to the palm assembly, wherein the coupling allows a wrist movement of the palm assembly in relation to at least one of the residual limb of the user and a forearm assembly, wherein the prosthetic device further comprises the forearm assembly, wherein the prosthetic device further comprises an actuator operationally coupled to each of the palm assembly and at least one of the wrist assembly, the residual limb of the user and the forearm assembly, wherein the actuator is configured to cause the wrist movement based on the action data.

6. The system of claim 1, wherein the prosthetic device comprises a forearm socket assembly and a wrist assembly, wherein a first end of the forearm socket assembly is configured to be coupled with the wrist assembly, wherein a second end of the forearm socket assembly is configured to be attached to the residual limb of the user.

7. The system of claim 1, wherein the prosthetic device comprises a forearm assembly and an upper socket assembly, wherein the forearm assembly is operationally coupled with the upper socket assembly, wherein the coupling allows a forearm movement of the forearm assembly in relation to the residual limb of the user, wherein an actuator is further configured to be operationally coupled with the forearm assembly and the upper socket assembly, wherein the actuator is configured to cause the forearm movement based on the action data.

8. The system of claim 1 further comprising an input device configured to receive a toggling input, wherein the toggling input is associated with an indication of selection of a mode from a plurality of modes, wherein the plurality of modes comprises a first mode in which the communication device is configured to transmit the action data to the controllable device, wherein the plurality of modes comprises a second mode in which the communication device is configured to transmit the action data to the prosthetic device, wherein the plurality of modes comprises a third mode in which the communication device is configured to transmit the action data to the controllable device and the prosthetic device.

9. The system of claim 1, wherein the action data corresponds to at least one of an application function performed by an external application executed on the controllable device and a device function provided by the controllable device.

10. The system of claim 9, wherein the communication device is configured to communicate with a user device comprising a user processing device configured to execute instructions corresponding to a management application, wherein the user device is further configured to present a user interface configured to receive an indication of an association between the action data and at least one of the application function and the device function.

11. The system of claim 1, wherein the analysis of the EMG data is performed using a machine learning model.

12. The system of claim 1, wherein the controllable device comprises one or more of a gamepad and a VR device.

13. The system of claim 1, wherein the controllable device is a computer device, wherein at least one of a motion and an action of a pointer in a graphical user interface of the computer device is controlled based on the action data.

14. The system of claim 1, wherein the communication device is a Bluetooth module.

15. The system of claim 1, wherein the communication device is further configured to receive a feedback data from the controllable device, wherein the feedback data is associated with a performance of the intended action by the controllable device, wherein the system further comprises a presentation device configured to present the feedback data.

16. The system of claim 15, wherein the presentation device comprises a haptic actuator configured to generate a tactile stimulus based on the feedback data, wherein the tactile stimulus is perceivable by the user.

17. The system of claim 16, wherein the presentation device comprises a light source configured to generate a visual signal based on the feedback data, wherein the visual signal is perceivable by the user.

18. The system of claim 1, wherein the communication device is further configured to communication with a user device comprising a user processing device, a user presentation device and a user communication device, wherein the user device is configured to execute instructions associated with a management application, wherein the management application is configure to present a plurality of user profile comprising a first user profile corresponding to a first functioning characteristics of the prosthetic device, wherein the plurality of user profile comprises a second user profile corresponding to a second functioning characteristics of the prosthetic device, wherein the user device is configured to receive a selection data associated with indication of selection of a user profile from a plurality of user profiles, wherein the communication device is configured to receive the user profile corresponding to the selection data, wherein the generation of the action data is based on the user profile.

19. The system of claim 1 further comprising an energy management device comprising an energy storage device and an energy management circuit, wherein the energy management device is coupled with the processing device, wherein the energy management circuit is configured to determine one or more of a energy storage device health parameter, a charge status associated with the energy storage device and a power consumption associated with the prosthetic device.

20. The system of claim 1, wherein the communication device is further configured to communicate with a user device, wherein analysis of EMG data is performed using a pre-trained machine learning model received from the user device, wherein the user device is configured to train the pre-trained machine learning model based on the EMG data received from the communication device.
